(19) United States
(12) Patent Application Publication
(10) Pub. No.: US 2007/0055076 A1
(43) Pub. Date: Mar. 8, 2007

(54) Processes for synthesizing esters by 1,4-addition of alkanoic acids to myrcene or isoprene

(76) Inventor: James H. Babler, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/564,307

(22) PCT Filed: Jul. 8, 2004
(86) PCT No.: PCT/US04/22075

§ 371 Date: Oct. 17, 2006

Related U.S. Application Data
(60) Provisional application No. 60/486,781, filed on Jul. 11, 2003.

(51) Int. Cl. C07C 67/02

(52) U.S. Cl. 560/249

(57) ABSTRACT

Processes are disclosed for synthesizing esters useful in flavorings and fragrances from β-pinene, myrcene and/or isoprene. The esters can be used in the manufacture of citral, precursors to citral and other products or precursors such as vitamins, nutritional supplements, flavorings, fragrances and other products. The process includes a 1,4-addition of an alkanoic acid to the conjugated diene of myrcene (which can be generated from β-pinene) or the conjugated diene of isoprene to produce esters thereof.

### RELATED APPLICATION

This application is based on U.S. Patent Application Ser. No. 60/486,781, filed Jul. 11, 2003, and claims priority therefrom.

### TECHNICAL FIELD

Processes are disclosed for synthesizing esters useful in flavorings and fragrances from β-pinene, myrcene and/or isoprene. The esters can be used in the manufacture of citral, precursors to citral and other products or precursors such as vitamins, nutritional supplements, flavorings, fragrances and other products.

### BACKGROUND OF THE RELATED ART

Citral is produced in large-scale processes since it is a raw material for the synthesis of many products including vitamins, other nutrients, flavorings and fragrances. Citral is widely used in the manufacture of important fragrances such as α- and β-ionones, and “methylionones.” As a result, thousands of tons of citral are manufactured each year.

α-Ionone is used in large quantities in the fragrance industry. β-Ionone is a costly specialty chemical that is used in the manufacture of vitamin A (widely used in the animal feed industry), the anti-acne drugs Retin-A™ and Accutane™, and several widely used carotenoids, including β-carotene and canthaxianthin. In addition to the thousands of tons of β-ionone that are required to manufacture these products each year, large quantities of β-ionone are used as an additive in pies, soaps, laundry-care products, household and beauty care products. β-ionone is also used in various flavorings.

In addition to citral, other commodity compounds used in manufacture of vitamins, flavorings and fragrances include prenyl acetate (3-methyl-2-buten-1-yl acetate), geranyl acetate [(2E)-3,7-dimethyl-2,6-octadien-1-yl acetate] and neryl acetate [(2Z)-3,7-dimethyl-2,6-octadien-1-yl acetate], related esters and the alcohols that these acetates are derived from. Prenyl, geranyl and neryl acetates are all precursors to citral and are valuable and versatile compounds in their own right.

However, the raw materials used to synthesize the precursors for citral and prenyl, geranyl and neryl acetates (and their related alcohols) are expensive and often involve toxic or corrosive reagents (e.g. alkyl halides and inorganic acids) or employ hazardous gases such as acetylene. The known manufacturing processes also generate large quantities of environmentally unfriendly waste.

Myrcene has been used as a starting material in the synthesis of citral or its precursors. Myrcene can be easily generated from β-pinene, which is a renewable natural product easily obtained from the volatile and extractable constituents of pine trees. However, the reaction of myrcene with an acid is known to result in products with a ring structure as shown below with the reaction labeled no. 2 below being the predominant mechanism: 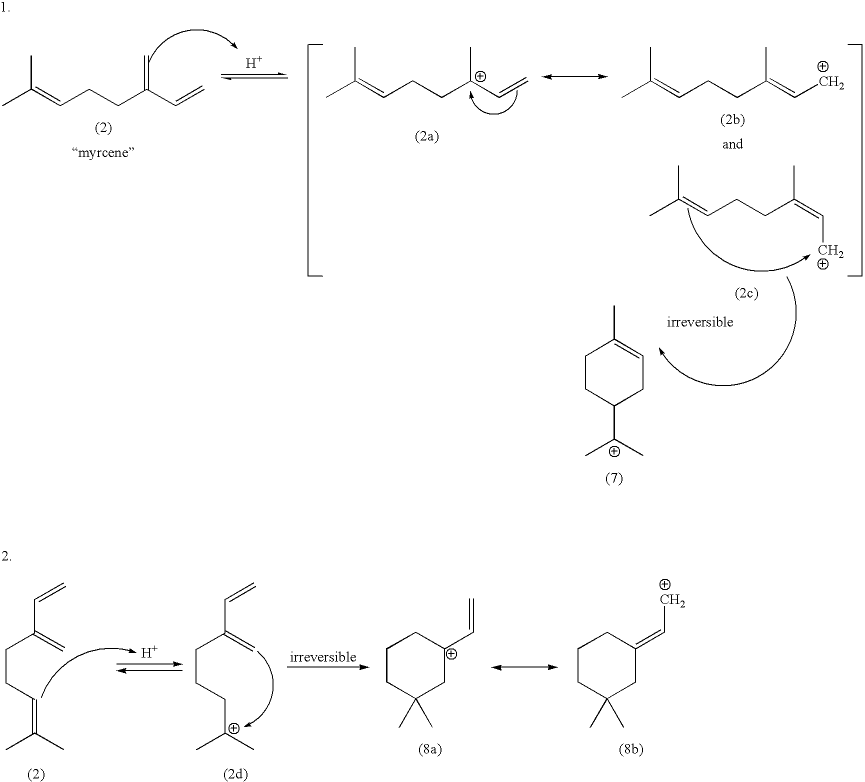
Indeed, this type of cyclization is expected in view of the fact that myrcene usually reacts with electrophilic reagents (e.g., acids) more rapidly at the isolated (i.e., non-conjugated) double bond. See B. Delmond et al., Synth. Commun., 23:2503 (1993).

Therefore, to generate useful ester materials and/or citral precursors from myrcene, gaseous HCl, a dangerous and toxic reagent, and a CuCl catalyst are used which create toxic allylic halide intermediates as shown below: 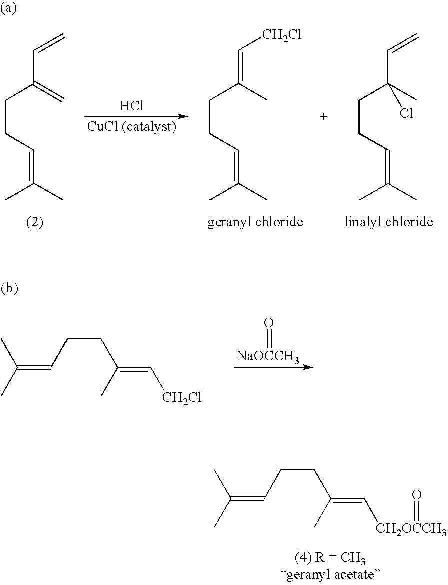
See, R. Weiss (International Flavors & Fragrances, Inc.), U.S. Pat. No. 2,882,323 (1959); Chem. Abstract, 53:17177 g (1959). In the absence of Cu⁺ salts, HCl attacks the non-conjugated double bond in myrcene as shown below: 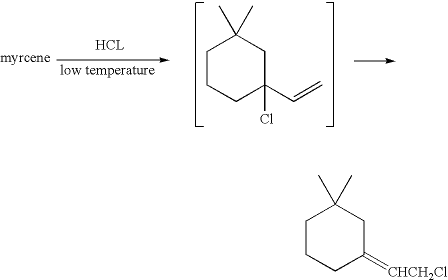
See, GB Patent No. 1,341,015 (1973).

Alkanoic acids such as acetic acid are known to add to the conjugated diene in myrcene but only in low yields (less than 20%). However, this reaction relies upon the presence of metallic Pd and cupric acetate under an oxygen atmosphere and the product mixture consists of both geranyl and neryl acetate, along with the less desirable and often unwanted linalyl acetate (i.e., a tertiary allylic acetate) which may need to be separated in a subsequent process. See, K. Suga et al., Yukagaku, 22:321 (1973), Chem. Abstracts, 79:665591 (1973).

With respect to the use of isoprene as a starting material for the fabrication of citral precursors or useful esters such as prenyl acetate, Julia et al., Bull. Soc. Chim. France, Part II, 588 (1980) teaches the following reaction: 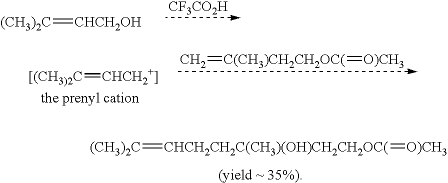
Thus, the literature teaches that the prenyl cation, generated in the presence of carboxylic acids, adds readily to the alkene functionality in unsaturated esters to give undesirable higher molecular weight products such as terpenes.

Accordingly, there is a need for an improved method for making citral, citral precursors, useful esters and other useful precursors including, but not limited to prenyl, geranyl and neryl esters that is relatively facile, that utilizes inexpensive and readily available starting materials, that avoids the use of toxic and corrosive reagents and that avoids the generation of toxic waste products or environmentally unfriendly waste products.

### SUMMARY OF THE DISCLOSURE

Novel processes are disclosed for preparing precursors to citral (3,7-dimethyl-2,6-octadienal) including but not limited to geranyl acetate [(2E)-3,7-dimethyl-2,6-octadien-1-yl acetate) and its stereoisomer neryl acetate [(2Z)-3,7-dimethyl-2,6-octadien-1-yl acetate] as well as prenyl acetate (3-methyl-2-buten-1-yl acetate) and related esters. It will be noted that geranyl, neryl and prenyl acetates are useful compounds apart from their use as citral precursors and the alcohols from which they are derived: geraniol [(2E)-3,7-dimethylocta-2,6-dien-1-ol]; nerol[(2Z)-3,7-dimethylocta-2,6-dien-1-ol]; and prenyl alcohol (3-methyl-2-buten-1-ol).

One disclosed process includes a 1,4-addition of an alkanoic acid (e.g., acetic acid, butyric acid, propionic acid or other carboxylic or alkanoic acid) to the conjugated diene in myrcene yielding ester derivatives of geraniol and nerol in an approximately 70:30 mixture of the two stereoisomers. The geranyl and neryl esters (or acetates) may then be converted to a mixture of geraniol and nerol, which are direct precursors to citral, or the geranyl and neryl esters may be used for other purposes.

Alternatively, another disclosed process may also include a 1,4-addition of an alkanoic acid to the conjugated diene in isoprene (2-methyl-1,3-butadiene), which yields ester derivatives of prenyl alcohol. The prenyl ester may then be converted to prenyl alcohol.

Thus, important precursor products or reagents are obtained by the 1,4 addition of alkanoic acids to the conjugated diene of myrcene or isoprene. The alkanoic acids are preferably C₂-C₈ liquid alkanoic acids, and having a Kₐ of preferably less than 10⁻⁴. Both myrcene and isoprene are inexpensive and safe starting materials. As noted above, myrcene can be easily generated from β-pinene, which is also inexpensive and abundant in supply.

Previously, it was thought by those skilled in the art that the 1,4-addition of an alkanoic or carboxylic acid to myrcene or isoprene was not possible. Due to the reactivity of cations resulting from the protonation of myrcene (see 2a in the flow chart above) or isoprene with an alkanoic acid such as acetic acid, such reactions should result in either a ring structure such as an acetate derivative of α-terpineol in the case of myrcene or undesirable higher molecular weight compounds such as terpenes in the case of isoprene as discussed in U.S. Pat. No. 5,872,277.

If one uses myrcene as a reagent in the disclosed processes, the only raw materials consumed during the synthetic scheme are β-pinene for generating the myrcene (or myrcene) and water as the alkanoic acid is recyclable. Likewise, if one uses isoprene as a reagent, the only raw materials consumed would be isoprene and water as the alkanoic acid is recyclable.

Although various alkanoic acids are utilized as a “reagent/solvent,” such alkanoic acids are cheap in addition to being renewable and indeed can be recovered/recycled after the enzyme-catalyzed hydrolysis of the initially-formed esters. All other reagents used are recyclable.

The disclosed processes also avoid the use of toxic or corrosive reagents. All synthetic steps of the disclosed processes involve relatively simple transformations and generate minimal waste. The disclosed processes avoid the use of acetylene and other hazardous gases. Further, the disclosed processes neither employ nor generate alkyl halides. Indeed, the disclosed processes and the manufacture of citral in accordance with this disclosure is environmentally friendly.

### DETAILED DESCRIPTION OF THE PRESENTLY PREFERRED EMBODIMENTS

### β-pinene or myrcene as the Primary Starting Material

As shown in the reaction flow charts below, the overall route to citral (6) that utilizes myrcene involves four steps, when starting with β-pinene (1) and three steps when starting with myrcene. The first step is the pyrolysis of β-pinene (1) to myrcene (2), is already safely practiced on an industrial scale and need not be discussed here.

The second step (or first step if the process begins with myrcene) involves a 1,4-addition of an alkanoic acid (e.g., acetic acid, propionic acid or other, carboxylic or alkanoic acid) to the conjugated diene in myrcene (2). Although the latter reaction appears to be similar to the addition of dichloroacetic acid (a costly raw material) to 2-methyl-1,3-butadiene (“isoprene”) as reported in U.S. Pat. No. 5,872,277, the following conversion of myrcene to geranyl ester (4) is a conceptually different (and considerably more difficult) reaction due to the presence of the isolated double bond in myrcene (2).

Indeed, in accordance with the prior art as discussed above, treatment of myrcene (2) with dichloroacetic acid, even in the presence of dichloroacetate as a buffer, at room temperature yielded a mixture of cyclized products with very little of the desired geranyl ester (4) where R═CHCl₂.

In sharp contrast, treatment of myrcene with refluxing acetic or alkanoic acid in accordance with the disclose processes (optionally in the presence of sodium acetate) affords a product mixture, the 400 MD ¹H NMR of which exhibited no absorption in the region of 0-1.3 ppm (delta scale). This NMR result is an indication that the isolated, non-conjugated double bond was not protonated under these conditions. Also remarkable about the disclosed process is that myrcene is essentially converted a 70:30 mixture of geranyl ester:neryl ester thereby minimizing the cyclic compounds derived from cation (7) as shown above in the background section of this disclosure.

The disclosed synthesis of neryl acetate (the acetate derivative of (2Z)-3,7-dimethylocta-2,6-dien-1-ol) and other neryl esters, is also remarkable and surprising in view of the fact that the intermediate cation, derived either by protonation of myrcene or by protonation and subsequent loss of water from “linalool,” has been shown to react in acetic acid to yield, as the major product, the acetate derivative of α-terpineol as set forth above and in J. H. Babler and D. O. Olsen, Tetrahedron Lett., 351-354 (1974) (see footnote no. 7 therein).

The preferred alkanoic acid is acetic acid (CH₃CO₂H). Use of less polar alkanoic acids such as propionic or isobutyric acid result in a slower process, albeit successful, and require higher internal temperatures to proceed at a reasonable rate. Under such conditions (e.g., 140-175° C.), pressure equipment is desirable to contain the myrcene (bp: 167° C.) in the liquid reaction mixture. Indeed, the best way to accelerate the reaction is by increasing the internal temperature. Although one can add small amounts of formic acid and sodium formate (in lieu of sodium acetate, et al.) to the mixture to accelerate the process, such a modification is not preferred due to the low solubility of myrcene in such a reaction mixture. Indeed, due to the limited solubility of myrcene in the “somewhat polar” acetic acid, mixtures of alkanoic acids, e.g., acetic acid along with some butyric acid (in which myrcene is very soluble), can also be used in the disclosed process.

Although mixtures of geranyl esters (see the structure labeled 4 below) are thereby obtained, such mixtures are subsequently hydrolyzed (see step (c) below) to yield the same alcohol (“geraniol”), which is the immediate precursor to “citral.” To dissolve even larger amounts of myrcene in this mixture of alkanoic acids (e.g., acetic acid and butyric acid), one can also add a non-basic organic co-solvent such as methylbenzene (“toluene”), 1,4-dimethylbenzene (“p-xylene”), butyl acetate, or butyl ether.

Another option to increase the solubility of myrcene in the reaction mixture (which is desirable, but not essential for the success of the procedure) is to add a non-basic organic co-solvent, e.g., butyl acetate, methoxybenzene (“anisole”), chlorobenzene, methylbenzene (toluene), butyl ether, or cyclohexanone, to the acetic acid. Indeed, by adding such co-solvents (butyric acid, “anisole,” etc.) to acetic acid, side-reactions resulting from the irreversible cyclization of cation (2c) to generate cation (7) (see the prior art discussion above) can be minimized.

Despite the teachings of the prior art, the applicant has found that the following reaction scheme for myrcene does work effectively as established by the results in Examples I through V below. 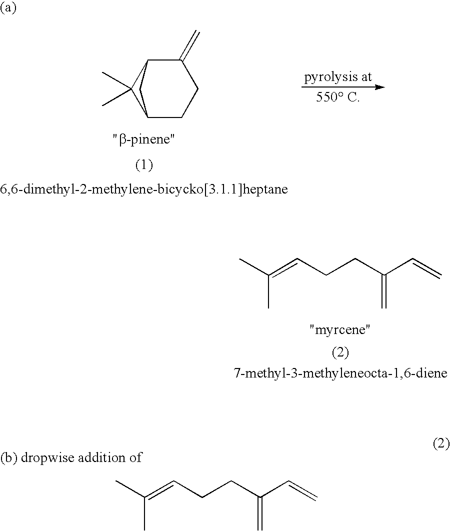
to a molar excess of a liquid alkanoic acid [RCO₂H, R═C₁-C₇ alkyl] in the presence of an alkanoate salt (e.g., sodium or potassium acetate), which is desirable but not essential for the transformation at a temperature greater than 100° C.;

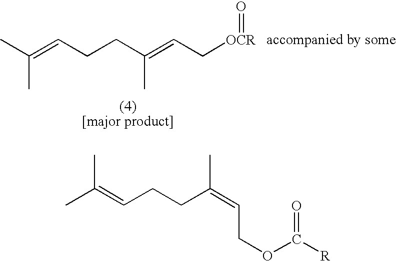

No solvent other than the alkanoic acid is required to conduct the reaction of step (b). Acetic acid (R═CH₃) is especially preferred in this process.

Since the process is slow at atmospheric pressure in the refluxing acetic acid (bp=118° C.), pressure equipment is desirable to obtain a higher internal temperature (e.g., 135-145° C.) and therefore a faster reaction. Another way to accelerate the reaction is to add to the mixture small amounts of formic acid and sodium formate (in lieu of sodium acetate, et al.) or benzoic acid (accompanied by some sodium benzoate). Mixtures of alkanoic acids, e.g., acetic acid along with some isobutyric acid, help to increase the solubility of myrcene.

Since geranyl acetate (4) and esters in general are quite stable under the reaction conditions (i.e., refluxing acetic acid), it might also be helpful to use such esters as a co-solvent to increase the solubility of myrcene in the reaction mixture.

When using higher-boiling alkanoic acids (e.g., isobutyric acid), it is also recommended that one use pressure equipment so that myrcene can be prevented from entering the vapor state (causing undesirable side-reactions).

The third and fourth stages (see steps c and d in the flow diagram below) of the disclosed procedure involve well-known transformations that result in high yields. 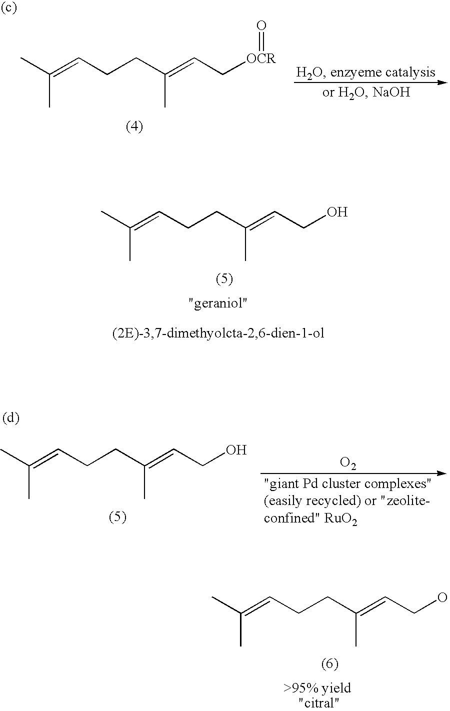
K. Kaneda, et al., Tetrahedron Lett., 38:9023 (1997) (use of “giant palladium cluster complexes”); M. A. White, et al., J. Am. Chem. Soc., 125:2195 (2003) (use of a zeolite-confined nanometer-sized RuO₂ catalyst).

It will be noted that geranyl esters, neryl esters, geraniol and nerol can be converted to citral but also are used as ingredients in perfumery. For example, geranyl isobutyrate (compound 4 above where R═CH(CH₃)₂) is a liquid with a fruity rose odor and is used in floral perfume compositions as well as in fruit flavors.

The following Examples I-V are presented for purposes of illustration using β-pinene or myrcene as the primary starting material and should not be construed as limiting this disclosure. Example VI illustrates an inferior prior process and Example VII is directed to the conversion of isoprene to prenyl acetate using a variation of the disclosed process.

### EXAMPLE I

### Preparation of Geranyl/Neryl Acetate by Treating Myrcene with Refluxing Acetic Acid

0.25 ml (1.47 mmoles) of myrcene (purchased from Aldrich Chemical Co., Milwaukee, Wis.), 5.0 ml of glacial acetic acid, and 51 mg (0.62 mmole) of anhydrous sodium acetate (purchased from Fisher Scientific Co.) were added to a 25-ml, 1-neck reaction flask containing a small spin bar and equipped with an efficient reflux condenser connected to an apparatus similar to that described by Johnson and Schneider [Org. Synth., 30:18 (1950)] so that the mixture in the flask could be protected from atmospheric conditions throughout the course of the reaction.

This mixture was subsequently heated at vigorous reflux (bp of acetic acid: 117° C. at atmospheric pressure) for 16 hours. For a large-scale process, it is recommended that myrcene be added dropwise to the heated reaction mixture.

After cooling the mixture to room temperature, the product was isolated by dilution of the reaction mixture with 50 ml of water and extraction with 40 ml of pentane. After subsequent washing of the organic layer with water (1×50 ml), 1M aqueous sodium hydroxide (1×30 ml), and saturated aqueous sodium chloride (1×25 ml), the washed organic layer was dried over anhydrous magnesium sulfate and filtered. Removal of pentane at reduced pressure (50-60 mm) afforded 162 mg of a product mixture which was characterized by proton No analysis (recorded in CDCl₃ solution at 400 MHz).

In Example I, as well as the other examples below conducted with acetic acid, very little, if any, absorption in the region of 0-1.3 ppm (delta scale) was observed—an indication that the isolated (i.e., non-conjugated) double bond in myrcene was not the favored site of protonation.

In Example I, trace amounts (less than 5% of the product mixture obtained from myrcene) of the latter type of undesirable by-products could be detected. Replacing approximately 20% of the acetic acid with organic co-solvent; such as butyl acetate, methoxybenzene, butyl ether, chlorobenzene suppressed that type of side-reaction that presumably arises from initial protonation of the non-conjugated double bond in myrcene. In an experiment similar to Example I, sodium acetate was not added to the reaction mixture, thereby resulting in an increase (approximately 10% of the product mixture obtained from myrcene) in the formation of the latter undesirable cyclic by-products. Furthermore, in the absence of sodium (or potassium) acetate, the formation of “alpha-terpinyl acetate” and “dipentene” (limonene) increased, although these latter two cyclic by-products (derived by initial protonation of the conjugated diene in myrcene and subsequent cyclization of that cation) were still minor components of the product mixture in most cases. Hence, although the presence of an alkanoate salt is not essential for this reaction, its presence in the reaction mixture is desirable.

The principal components in the product mixture were “unreacted” myrcene, geranyl acetate and the “stereoisomeric” neryl acetate, limonene (“dipentene”), and alpha-terpinyl acetate. Due to its volatility (bp: 167° C. at atmospheric pressure), some of the “unreacted” myrcene was lost during the removal of pentane in the product isolation procedure, thereby explaining the failure to obtain a good material balance in these experiments. Proton NMR spectra of authentic samples of each of these compounds were recorded and used to confirm the presence of each in the product mixture. Integration of selected signals (cited below) on the proton NMR spectrum of the product mixture led to a determination of the composition of this mixture. In a subsequent experiment, the % conversion of myrcene to geranyl/neryl acetate by use of NMR data was further verified by chromatographic separation of the product mixture to obtain a purified sample of “geranyl/neryl” acetate.

In Example I, approximately 60% of the “crude product mixture” consisted of unreacted myrcene. The % conversion of starting myrcene to the desired geranyl/neryl acetate was approximately 17% (i.e., approximately 40% yield, based on unreacted myrcene). The other principal component in this reaction product mixture was limonene (“dipentene”)—the formation of which could be significantly reduced by the addition of various organic co-solvents to the reaction mixture, thereby resulting in increased yields of geranyl/neryl acetate of up to approximately 75%.

The % conversion of myrcene to geranyl/neryl acetate was rather low (10-20%) when these experiments were conducted at 117° C. at atmospheric pressure. However, the rate of this reaction was shown to be significantly increased by raising the reaction temperature. For example, an experiment similar to the one described in Example I was conducted at 95° C. (external oil bath temperature) and resulted in little (if any) (<2%) formation of the desired geranyl/neryl acetate after 15 hours. In order to achieve a reasonable rate of reaction, the acetic acid/myrcene mixture should be heated at a temperature in the range of 140° C., using pressure equipment.

Representative proton NMR data for the components in the crude product mixture of Example I are:

(a) geranyl acetate: CH₃C═O: singlet at δ 2.046

CH₂O: doublet (J=7.2 Hz) at δ 4.58

(b) neryl acetate:

CH₃C═O: singlet at δ 2.040

CH₂O: doublet (J=7.2 Hz) at δ 4.55

The ratio of the above 2 stereoisomers was approximately 70:30 in virtually all experiments.

(c) alpha-terpinyl acetate:

C(CH₃)₂: singlet (6H's) at δ 1.42

CH₃C═O: singlet (3 Hrs) at δ 1.95

(d) limonene (“dipentene”):

C═CH₂: broad singlet at δ 4.70

(e) myrcene: vinyl H bonded to ° C-2: an “AB quartet” (4 lines) centered at δ 6.37.

### EXAMPLE II

### Preparation of Geranyl/Neryl Acetate by Treatment of Myrcene with Refluxing Acetic Acid in the Presence of a Non-Basic Organic Co-Solvent

Using a procedure similar to that described in Example I, a mixture of 0.25 ml (200 mg, 1.47 mmoles) of myrcene, 1.00 ml of chlorobenzene (spectrophotometric-grade), 4.00 ml of glacial acetic acid, and 91 mg (1.11 mmoles) of anhydrous sodium acetate was heated at vigorous reflux (atmospheric pressure) for 20 hours. After cooling the mixture to room temperature, the product was isolated as described in the procedure of Example I. In order to remove chlorobenzene from this product, it was subjected to reduced pressure (as low as 1 mmHg) for approximately 10-15 minutes. N analysis of this product as described in Example I indicated that the process was somewhat slower in this presence of a co-solvent, i.e., approximately 10% conversion of myrcene to geranyl/neryl acetate. However, other by-products were formed to a lesser-extent and the % yield based on unreacted myrcene) now exceeded 60%.

In an identical experiment that was conducted for 40 hours, the % conversion of myrcene to a 70:30 mixture of geranyl/neryl acetate was approximately 18%, an indication that pressure equipment and higher temperature will be desirable if one wants to reduce the reaction time. More significantly, the ratio of products (e.g., 6:1 mixture of geranyl/neryl acetate: alpha-terpinyl acetate) was virtually identical in the two experiments; an indication that under these conditions the desired product is reasonably stable.

Similar results were obtained by using various other co-solvents (butyl acetate, butyl ether, p-xylene, methyl isobutyl ketone, etc.) in lieu of chlorobenzene.

### EXAMPLE III

### Preparation of Geranyl/Neryl Acetate by Treating Myrcene with Refluxing Acetic Acid in the Presence of Anisole

Using a procedure similar to that described in Example I, a mixture of 0.25 ml (200 mg, 1.47 mmoles) of myrcene, 1.00 ml of methoxybenzene (“anisole,” purchased from Fisher Scientific Co.), 4.00 ml of glacial acetic acid, and 97 mg (1.18 mmoles) of anhydrous sodium acetate was heated at vigorous reflux (atmospheric pressure) for 18 hours. After cooling the mixture to room temperature, the product was isolated as described in the procedure of Example I. Most of the anisole (along with some of the “unreacted” myrcene) was removed at reduced pressure (as low as 1 mm) over a period of approximately 40 minutes.

The residual material (200 mg) was then chromatographed on 15 ml of “Baker-Analyzed” silica gel (40-140 mesh). After removal of unreacted myrcene and traces of anisole by elution with pentane (50 ml), the desired acetate esters were, washed off the column using 45 ml of 2:1 (v/v) pentane:ether. Removal of the pentane and ether at reduced pressure (60 mm) and subsequent evaporative (“Kugelrohr” oven) distillation afforded 36 mg (12% conversion) of a 6:1 mixture of geranyl/neryl:alpha-terpinyl acetates (determined by proton NMR analysis and comparison with authentic samples). The boiling point of this latter product was 68-75° C. (bath temperature, 0.50 mm).

A similar experiment was conducted for which sodium acetate was replaced by an equivalent amount of potassium acetate. The product mixtures obtained in both experiments were virtually identical. Consistent with the experimental results described in Example I, a similar experiment using a 4:1 volume ratio of acetic acid:anisole was conducted in the absence of any acetate salt which resulted in additional by-products, e.g., more alpha-terpinyl acetate.

Although sodium or potassium acetate is not essential for the success of the desired conversion (i.e., myrcene→geranyl/neryl acetate), its presence in the reaction mixture is desirable.

### EXAMPLE IV

### Preparation of Geranyl/Neryl Propionate by Treating Myrcene with Refluxing Propionic Acid in the Presence of Chlorobenzene

Using a procedure similar to that described in Example L a mixture of 0.25 ml (200 mg, 1.47 mmoles) of myrcene, 1.00 ml of chlorobenzene (spectrophotometric-grade, bp: 132° C.), 4.00 ml of propionic acid (99% pure; bp: 141° C.; purchased from Aldrich Chemical Company, Milwaukee, Wis.), and 110 mg (1.15 mmole) of sodium propionate (purchased from Fisher Scientific Co.) was heated at vigorous reflux (atmospheric pressure) for 17 hours. After cooling the mixture to room temperature, the product was isolated as described in the procedure of Example I. In order to remove chlorobenzene from this product, it was subjected to reduced pressure (approximately 1 mm) for 10-15 minutes, after which 125 mg of crude product mixture was recovered and subjected to proton NMR analysis (400 MHz).

The NMR results indicate the absence of by-products obtained by protonation of the isolated (i.e., non-conjugated) double bond in myrcene, i.e., no absorption in the region of 0-1.1 ppm (delta scale). Most of this crude product mixture consisted of “unreacted” myrcene (some of which was also undoubtedly lost during the removal of chlorobenzene at reduced pressure. Although this example was conducted at higher temperature (135-140° C.) than the process described in Example II, the lower polarity of propionic acid (vs. acetic acid) resulted in a slower reaction; and the % conversion of myrcene to the desired geranyl/neryl propionate was only 6%.

The identity of this product was ascertained by comparison of its proton NMR spectrum with that exhibited by authentic samples of geranyl propionate and its stereoisomer, neryl propionate. The proton NMR spectrum of geranyl propionate exhibited a triplet (J=7.6 Hz) at δ 1.14 (CH₃CH₂C═O), a quartet (J=7.6 Hz) at δ 2.325 (CH₃CH₂C═O), a doublet (J=7.2 Hz) at δ 4.596 (CH₂O), and a broad triplet (J=7.2 Hz) at δ 5.34 (vinyl H on the C₂-C₃ double bond). Neryl propionate was characterized by a doublet (J=7.6 Hz) at δ 4.567 (CH₂O).

### EXAMPLE V

### Preparation of Geranyl/Neryl Esters by Treating Myrcene with a Mixture of Alkanoic Acids

Using a procedure similar to that described in Example I, a mixture of 0.25 ml (200 mg, 1.47 mmoles) of myrcene, 1.00 ml of methoxybenzene (“anisole,” purchased from Fisher Scientific Co.), 1.00 ml (9.2 mmoles) of isovaleric acid purchased from Aldrich Chemical Co., Milwaukee, Wis.), 3.00 ml (52.4° mmoles) of glacial acetic acid, and 96 mg (1.17 mmoles) of anhydrous sodium acetate was heated at vigorous reflux (atmospheric pressure) for 18 hours. After cooling the mixture at room temperature, the product was isolated as described in the procedure of Example I. Most of the anisole (along with some of the “unreacted” myrcene) was removed at reduced-pressure (as low as 1 mmHg) over a period of approximately 40 minutes.

The residual material (196 mg), which contained traces of anisole along with unreacted myrcene, was subjected to analysis by recording a proton NMR spectrum of this mixture (CDCl₃ solution, 400 MHz) and integrating the areas of signals arising from each of the components known to be in the mixture. This analysis indicated that the reaction proceeded more slowly in the presence of both a co-solvent and the less polar (vs. acetic acid) C-5 alkanoic acid, i.e., approximately 8% conversion of myrcene to a 6:1 mixture of geranyl/neryl acetate:geranyl/neryl isovalerate. The latter ester was characterized by a doublet (J=6.4 Hz) at δ 0.94 [HC (CH₃)₂, 6H's] and a doublet (J=7.6 Hz) at δ 4.59 (CH₂O). Although the process was slow, by-products such as alpha-terpinyl esters and limonene (“dipentene”) were formed to a lesser extent in the less polar reaction mixture.

Although the above experimental conditions result in the formation of two geranyl esters (geranyl acetate and geranyl isovalerate), if one intends to manufacture geraniol, and subsequently oxidize it to citral, such a mixture does not have to be separated. Both esters can be simultaneously hydrolyzed by water in the presence of a lipase enzyme to yield geraniol and a mixture of water-soluble alkanoic acids.

### EXAMPLE VI (PRIOR ART)

### Treatment of Myrcene with Dichloroacetic Acid

324 mg (1.94 mmoles) of potassium dichloroacetate (purchased from Aldrich Chemical Co., Milwaukee, Wis.) and 4.00 ml of dichloroacetic acid (purified-grade, purchased from Fisher Scientific Co.) were added to a 25 ml, 3-neck reaction flask fitted with a septum cap (to allow addition of myrcene to be made using a syringe) and an adapter connected to an apparatus similar to that described by Johnson and Schneider [Org. Synth., 30:18 (1950)] so that the mixture in the flask could be protected from atmospheric moisture throughout the course of the reaction.

The mixture was stirred for several minutes at room temperature until all solid had dissolved, after which the reaction was initiated by addition of 60 microliters (μl) of myrcene to the stirred-reaction mixture. Every 15 minutes, an additional portion (60 μl) of myrcene was added until three such portions (3×60 μl; 1.06 mmoles) of myrcene had been added over a period of 30 minutes. The mixture was subsequently stirred at room temperature for an additional 15 minutes. The product was isolated by dilution of the reaction mixture with 40 ml of 10% (w/v) aqueous sodium chloride and extraction with hexane (1×30 ml). After subsequent washing of the organic layer with saturated aqueous sodium bicarbonate (1×25 ml) and saturated aqueous sodium chloride (1×25 ml), the organic layer was dried over anhydrous magnesium sulfate and filtered. Removal of the hexane by evaporation at reduced pressure afforded 205 mg of a crude mixture of products, greater than 75% of which had been formed by protonation of the isolated (i.e., non-conjugated) double bond in myrcene.

Proton NMR analysis (400 MHz) of the above product mixture indicated that geranyl/neryl dichloroacetate comprised less than 20% of the product mixture since the absorption peaks in the δ 1.55-1.80 region (due to “vinyl CH₃'s”) were quite small. Several strong signals in the δ 0.85-0.90 region (CH₃'s bonded to a 6-membered ring) verified that myrcene had been protonated at the non-conjugated double bond to generate a cation, which rapidly underwent cyclization to afford products possessing a 6-membered ring. The results are similar to that reported in GB Patent No. 1,341,015 (1973) in regard to the addition of HCl to myrcene.

### Use of Isoprene as a Primary Starting Material

Similar reaction conditions can also be used to convert isoprene to prenyl acetate. Thus one can add CH₂═C(CH₃)CH═CH₂ (isoprene) dropwise to a pressure vessel containing a molar excess of glacial acetic acid, and optionally some acetate salt (e.g., sodium acetate) at temperatures exceeding 100° C. (e.g., 140° C.). To increase the solubility of isoprene in the reaction mixture, one can also add a non-basic organic co-solvent (e.g., one or more aromatic hydrocarbons such as toluene, esters such as isopropyl acetate, or ketones) to the reaction mixture.

The reaction of isoprene with acetic acid, which occurs slowly, is not problematic as isoprene is quite soluble in acetic acid, even in the presence of sodium acetate. In a solution of approximately 0.1-0.2 M NaOAc in acetic acid, isoprene will be converted to prenyl acetate (very high purity unless the isoprene concentration is too large) at a reasonable rate at temperatures of about 140° C. Product isolation is not difficult: The mixture is cooled and diluted with an alkane thereby leaving behind the acetate salt. After that, one can fractionally distill the liquid mixture or (more conveniently) wash out the acetic acid with water.

The product of the above transformation is (CH₃)₂C═CHCH₂C(═O)CH₃ (prenyl acetate), which is used in the flavor and fragrance industries. These results and the reactions presented below are unexpected based on results reported in U.S. Pat. Nos. 5,872,277 and 6,278,016 both of which teach that such a process requires more acidic conditions.

Further, the literature suggests that, at higher reaction temperatures, if acetic acid is capable of protonating isoprene (to generate the “prenyl cation”), under such vigorous reaction conditions, further transformations of the initially-formed prenyl acetate (9) to higher-molecular-weight terpenoids would result. Thus, the disclosed process involves protonation of isoprene to yield the prenyl cation: (CH₃)₂C═CHCH₂⁺. This cation is known to react with unsaturated esters, (such as CH₂═C(CH₃)CH₂CH₂C(═O)CH₃] similar to the structure of prenyl acetate (9). See Table X, entries 5 and 7 on page 595 in an article by Julia et al., Bull. Soc. Chim. France, Part II, 588(1980).

Since the disclosed process involves both the prenyl cation (generated by protonation of isoprene) and unsaturated esters [i.e., formation of prenyl acetate (9)], the formation of prenyl acetate (9) in high yields was suprising in view of the contrary teachings of the prior art. In contrast to the results provided herein, one would expect subsequent reaction of the initially-formed prenyl acetate (9), especially at high temperatures in the presence of a carboxylic acid, to re-generate the prenyl cation, eventually leading to dimeric products.

An important use of prenyl acetate would be its hydrolysis to prenyl alcohol and subsequent oxidation of the latter alcohol to 3-methyl-2-butenal (renal). Prenyl alcohol and prenal can be used in a “one-pot” process to obtain “citral” in high yield. Chem. Abstracts, 112:179516d (1990); Chem. Abstracts, 115:114815t (1991); U.S. Pat. No. 5,872,277.

The treatment of isoprene with an alkanoic acid proceeds as follows: 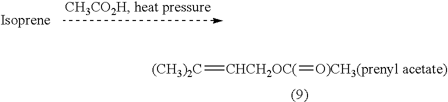
In a similar process, isoprene can be treated with a molar excess of propionic acid to yield prenyl propionate. 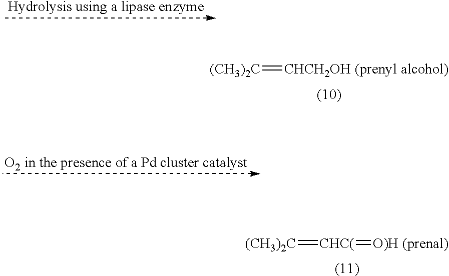
The hydrolysis using a lipase enzyme is discussed in T. Itoh et al., Tetrahedron Lett., 37:91 (1996); the oxidation of prenyl alcohol is discussed in K. Kaneda et al., J. Org. Chem., 61:4502 (1996); the oxidation of prenyl alcohol in air and in the presence of various metallic salt catalysts to yield the corresponding aldehyde (prenal (11)) is discussed in M. Matsumoto et al., J. Org. Chem., 49:3435 (1984), Japanese Patent 60 239,443; and Chem. Abstracts, 104:148312q (1986).

Another approach to the formation of prenyl alcohol (10) from isoprene involves the addition of hydrohalic acids (HX: HCl or HBr) to isoprene. Although this reaction does yield prenyl halides ((12) (CH₃)₂C═CHCH₂X, X═Br or C1), yields are only moderate and the reaction is complicated by the fact that HX also adds to the double bond in the initially formed prenyl halide to give a dihalide, (CH₃)₂C(X)CH₂CH₂X. Furthermore, prenyl bromide (or chloride) is highly toxic, volatile, and decomposes if one attempts to distill it at atmospheric pressure. On the other hand, if one has prenyl halides (12) available, the following route to prenyl alcohol has been developed: 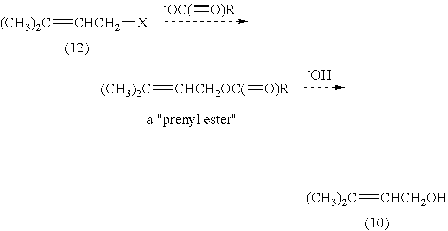
See Japanese Patent No. 77-10,207; Chem. Abstracts, 87:38852p (1977); German Patent No. 3021414; Chem. Abstracts, 94:174311h (1981).

The following Example VII provides one procedure for treating isoprene with an alkanoic acid to produce the useful material prenyl acetate.

### EXAMPLE VII

### Preparation of 3-Methyl-2-buten-1-yl Acetate (“Prenyl Acetate”) by Treating Isoprene with Excess Acetic Acid

0.50 ml (5.0 mmoles) of isoprene (purchased from Aldrich Chemical Co., Milwaukee, Wis.), 12.0 ml of glacial acetic acid, and 144 mg (1.76 mmoles) of anhydrous sodium acetate were added to a 15 ml pressure vessel (heavy glass wall, purchased from Chemglass, Vineland; N.J.). During a large-scale process, it is recommended that isoprene be added slowly to the pressurized heated reaction mixture.

After adding a small spin bar, the pressure vessel was closed; and the mixture was heated, with continuous stirring, at 125° C. (external oil bath temperature) for 16 hours. After cooling the mixture to room temperature, the product was isolated by dilution of the reaction mixture with 120 ml of water and extraction with pentane. After subsequent washing of the organic layer with water (100 ml), saturated aqueous sodium bicarbonate (1×50 ml), and saturated aqueous sodium chloride (1×25 ml), the organic layer was dried over anhydrous magnesium sulfate and filtered. Removal of most of the pentane by fractional distillation at atmospheric pressure, followed by removal of residual pentane at reduced pressure (60 mmHg) afforded 92 mg (15% conversion but >90% yield if one considers the large amount of unreacted isoprene when the process is conducted at such a low temperature) of the named ester, and substantially free of impurities.

The identity and purity of the product in this reaction was ascertained by IR and proton NMR analysis (recorded at 300 MHz in CDCl₃ solution). The NMR spectrum shows a broad triplet (J=7.2 Hz) at δ 5.35 (CH═C), a doublet (J=7.2 Hz) at δ 4.57 (CH₂O), a singlet at δ 2.05 (CH₃C═O), and signals for two vinyl methyls at δ 1.76 and 1.71.

Due to the limited solubility of isoprene in acetic acid containing small amounts of sodium acetate, it may be desirable to add minor amounts (approximately 15-20%, on a volume basis) of non-basic organic co-solvents (e.g., esters such as ethyl or isopropyl acetate, or aromatic hydrocarbons such as toluene) to the reaction mixture.

Regarding such non-basic co-solvents, the conjugate acid (i.e., protonated form) of such solvents should have a pKa (relative to water) of “−2” or a larger-negative number (e.g., −6). Thus, the pKa of the conjugate acid of methoxybenzene (“anisole”) is approx. “−6” and of cyclohexanone is approx. “−7.”

Although the foregoing processes have been described in some detail by way of illustration and examples for purposes of clarity and understanding, it will be apparent to those skilled in the art that various changes and modifications may be practical. Therefore, the description and examples set forth above should not be construed as limiting the scope of this disclosure or the appended claims.

1.-44. (canceled)

45. A method for preparing an ester from a conjugated diene compound selected from the group consisting of myrcene, isoprene, and mixtures thereof, the method comprising:
providing a solution containing at least one alkanoic acid of the formula R₁ CO₂H wherein R₁ is a C₁ to C₇ alkyl group and having a Kₐ relative to water of less than 10⁻⁴;
heating the solution to a temperature in excess of 100° C.; and
adding the conjugated diene compound to the solution to form a reaction mixture free of catalysts while maintaining said alkanoic acid in a molar concentration greater than that of the conjugated diene compound, to produce an ester derivative of the conjugated diene compound.

46. The method of claim 45, wherein the conjugated diene compound is myrcene.

47. The method of claim 45, wherein the conjugated diene compound is isoprene.

48. The method of claim 47, wherein the alkanoic acid is acetic acid.

49. The method of claim 45, wherein the conjugated diene compound is added to the liquid reaction mixture in a dropwise fashion.

50. The method of claim 45, wherein the solution comprises a mixture of alkanoic acids.

51. The method of claim 50, wherein the mixture of alkanoic acids includes acetic acid and the conjugated diene compound includes myrcene.

52. The method of claim 51, wherein the mixture of alkanoic acids further includes an acid selected from the group consisting of butyric acid, isobutyric acid, and combinations thereof.

53. The method of claim 52, wherein the solution further comprises a non-basic organic co-solvent selected from the group consisting of methylbenzene, butyl ether, chlorobenzene, 1,4-dimethylbenzene, methoxybenzene, cyclohexanone, butyl acetate and mixtures thereof.

54. The method of claim 45, wherein the liquid solution further comprises a base having a formula (R₂CO₂)M wherein R₂ is C₁ to C₇ alkyl, M is a group I cation and R₂ can be the same or different than R₁.

55. The method of claim 54, wherein the base is selected from the group consisting of sodium acetate, potassium acetate and sodium propionate.

56. The method of claim 45, comprising conducting the reaction in a pressurized vessel.

57. The method of claim 56, wherein the reaction mixture is maintained at a temperature in a range of about 115° C. to about 175° C. during and after the adding of the conjugated-diene compound.

58. The method of claim 57, wherein the temperature is in a range of about 135° C. to about 145° C. and the conjugated diene compound is myrcene.

59. The method of claim 45, wherein R₁CO₂H is selected from the group consisting of acetic acid, propionic acid, butyric acid, isobutyric acid, isovaleric acid and mixtures thereof.

60. The method of claim 45, wherein said solution further comprises a non-basic organic co-solvent.

61. The method of claim 60, wherein the non-basic organic co-solvent is selected from the group consisting of ethyl acetate, isopropyl acetate, 2-butanone, methylbenzene and mixtures thereof.

62. The method of claim 61, wherein the conjugated diene compound is isoprene.

63. A method for preparing geranyl and neryl esters from myrcene, comprising:
providing a solution comprising
a mixture of alkanoic acids comprising acetic acid and a one or more additional alkanoic acids selected from the group consisting of propionic acid, butyric acid, isobutyric acid, isovaleric acid and mixtures thereof,
a non-basic organic co-solvent selected from the group consisting of methylbenzene, butyl ether, chlorobenzene, 1,4-dimethylbenzene, methoxybenzene, cyclohexanone, butyl acetate and mixtures thereof, and
a base selected from the group consisting of sodium acetate, potassium acetate, sodium propionate, and mixtures thereof;
heating the solution in a pressurized vessel to a temperature in excess of 100° C.; and
adding the myrcene to the alkanoic acids in a dropwise fashion to form a reaction mixture free of catalysts while maintaining said alkanoic acids in a molar concentration greater than that of the myrcene, to produce a geranyl ester/neryl ester mixture.

64. A method for preparing a prenyl ester from isoprene, comprising:
providing a solution comprising
an alkanoic acid comprising acetic acid and optionally one or more additional alkanoic acids of the formula R₁CO₂H wherein R₁ is a C₁ to C₇ alkyl group and having a Kₐ relative to water of less than 10⁻⁴,
a non-basic organic co-solvent selected from the group consisting of methylbenzene, ethyl acetate, isopropyl acetate, 2-butanone, and mixtures thereof, and
a base selected from the group consisting of sodium acetate, potassium acetate, sodium propionate, and mixtures thereof;
heating the solution in a pressurized vessel to a temperature in excess of 100° C.; and
adding the isoprene to the alkanoic acid in a dropwise fashion to form a reaction mixture free of catalysts while maintaining said alkanoic acid in a molar concentration greater than that of the isoprene, to produce a prenyl ester.
